(19) United States
(12) Patent Application Publication
(10) Pub. No.: US 2003/0171808 A1
(43) Pub. Date: Sep. 11, 2003

(54) Accommodating intraocular lens

(76) Inventor: Andrew F. Phillips, Pasadena, CA (US)

Correspondence Address: David P. Gordon, 65 Woods End Road, Stamford, CT, 06905, US

(21) Appl. No.: 10/090,675

(22) Filed: Mar. 5, 2002

(51) Int. Cl. A61F 2/16

(52) U.S. Cl. 623/6.37; 623/6.45

(57) ABSTRACT

An intraocular lens (IOL) system includes a flexible lens having a skirt, and a restraining element. The restraining element is provided about the skirt and maintains the flexible lens in a stressed, non-accommodating configuration. At implantation, the ciliary body muscle is pharmacologically induced into a relaxed state, a capsulorrhexis is performed on the lens capsule, and the natural lens is removed from the capsule and replaced with the lens of the invention. The ciliary body is maintained in the relaxed state for several weeks until the capsule heals and shrinks about the lens. The restraining element is preferably designed to be removed in a non-surgically invasive manner, e.g., via dissolution or laser light, after capsule healing has occurred. The ciliary body and lens may then interact in a manner substantially similar to the physiological interaction between the ciliary body and a healthy natural crystalline lens.

### BACKGROUND OF THE INVENTION

1. Field of the Invention

This invention relates broadly to ophthalmic implants. More particularly, this invention relates to intraocular lenses which are focusable and allow for accommodation for near vision.

2. State of the Art

Referring to FIG. 1, the human eye 10 generally comprises a cornea 12, an iris 14, a ciliary body (muscle) 16, a capsular bag 18 having an anterior wall 20 and a posterior wall 22, and a natural crystalline lens 24 contained with the walls of the capsular bag. The capsular bag 18 is connected to the ciliary body 16 by means of a plurality of zonules 26 which are strands or fibers. The ciliary body 16 surrounds the capsular bag 18 and lens 24, defining an open space, the diameter of which depends upon the state (relaxed or contracted) of the ciliary body 16.

When the ciliary body 16 relaxes, the diameter of the opening increases, and the zonules 26 are pulled taut and exert a tensile force on the anterior and posterior walls 20, 22 of the capsular bag 18, tending to flatten it. As a consequence, the lens 24 is also flattened, thereby undergoing a decrease in focusing power. This is the condition for normal distance viewing. Thus, the emmetropic human eye is naturally focussed on distant objects.

Through a process termed accommodation, the human eye can increase its focusing power and bring into focus objects at near. Accommodation is enabled by a change in shape of the lens 24. More particularly, when the ciliary body 16 contracts, the diameter of the opening is decreased thereby causing a compensatory relaxation of the zonules 26. This in turn removes or decreases the tension on the capsular bag 18, and allows the lens 24 to assume a more rounded or spherical shape. This rounded shape increases the focal power of the lens such that the lens focuses on objects at near.

As such, the process of accommodation is made more efficient by the interplay between stresses in the ciliary body and the lens. When the ciliary body relaxes and reduces its internal stress, there is a compensatory transfer of this stress into the body of the lens, which is then stretched away from its globular relaxed state into a more stressed elongated conformation for distance viewing. The opposite happens as accommodation occurs for near vision, where the stress is transferred from the elongated lens into the contracted ciliary body.

In this sense, referring to FIG. 2, there is conservation of potential energy (as measured by the stress or level of excitation) between the ciliary body and the crystalline lens from the point of complete ciliary body relaxation for distance vision through a continuum of states leading to full accommodation of the lens.

As humans age, there is a general loss of ability to accommodate, termed “presbyopia”, which eventually leaves the person unable to focus on near objects. In addition, when cataract surgery is performed and the natural crystalline lens is replaced by an artificial intraocular lens, there is generally a complete loss of the ability to accommodate. This occurs because the active muscular process of accommodation involving the ciliary body is not translated into a change in focusing power of the implanted artificial intraocular lens.

There have been numerous attempts to achieve at least some useful degree of accommodation with an implanted intraocular lens which, for various reasons, fall short of being satisfactory. In U.S. Pat. No. 4,666,446 to Koziol et al., there is shown an intraocular lens having a complex shape for achieving a bi-focal result. The lens is held in place within the eye by haptics which are attached to the ciliary body. However, the implant requires the patient to wear spectacles for proper functioning. Another device shown in U.S. Pat. No. 4,944,082 to Richards et al., also utilizes a lens having regions of different focus, or a pair of compound lenses, which are held in place by haptics attached to the ciliary body. In this arrangement, contraction and relaxation of the ciliary muscle causes the haptics to move the lens or lenses, thereby altering the effective focal length. There are numerous other patented arrangements which utilize haptics connected to the ciliary body, or are otherwise coupled thereto, such as are shown in U.S. Pat. No. 4,932,966 to Christie et al., U.S. Pat. No. 4,888,012 to Horne et al. and U.S. Pat. No. 4,892,543 to Turley, and rely upon the ciliary muscle to achieve the desired alteration in lens focus.

In any arrangement that is connected to the ciliary body, by haptic connection or otherwise, extensive erosion, scarring, and distortion of the ciliary body usually results. Such scarring and distortion leads to a disruption of the local architecture of the ciliary body and thus causes failure of the small forces to be transmitted to the intraocular lens. Thus, for a successful long-term implant, connection and fixation to the ciliary body is to be avoided if at all possible.

In U.S. Pat. No. 4,842,601 to Smith, there is shown an accommodating intraocular lens that is implanted into and floats within the capsular bag. The lens comprises front and rear flexible walls joined at their edges, which bear against the anterior and posterior inner surfaces of the capsular bag. Thus, when the zonules exert a tensional pull on the circumference of the capsular bag, the bag, and hence the intraocular lens, is flattened, thereby changing the effective power of refraction of the lens. The implantation procedure requires that the capsular bag be intact and undamaged and that the lens itself be dimensioned to remain in place within the bag without attachment thereto. Additionally, the lens must be assembled within the capsular bag and biasing means for imparting an initial shape to the lens must be activated within the capsular bag. Such an implantation is technically quite difficult and risks damaging the capsular bag, inasmuch as most of the operations involved take place with tools which invade the bag. In addition, the Smith arrangement relies upon pressure from the anterior and posterior walls of the capsular bag to deform the lens, which requires that the lens be extremely resilient and deformable. However, the more resilient and soft the lens elements, the more difficult assembly within the capsular bag becomes. Furthermore, fibrosis and stiffening of the capsular remnants following cataract surgery may make this approach problematic.

U.S. Pat. No. 6,197,059 to Cumming and U.S. Pat. No. 6,231,603 to Lang each disclose an intraocular lens design where the configuration of a hinged lens support ostensibly allows the intraocular lens to change position in response to accommodation and thus change effective optical power. U.S. Pat. No. 6,299,641 to Woods describes another intraocular lens that also increases effective focusing power as a result of a change in axial position during accommodation. In each of these intraocular lenses, a shift in axial position and an increase in distance from the retina results in a relative increase in focusing power. All lenses that depend upon a shift in the position of the lens to achieve some degree of accommodation are limited by the amount of excursion possible during accommodation.

U.S. Pat. No. 5,607,472 to Thompson describes a dual-lens design. Prior to implantation, the lens is stressed into a non-accommodative state with a gel forced into a circumferential expansion channel about the lens. At implantation, the surgeon must create a substantially perfectly round capsullorrhexis, and insert the lens therethrough. A ledge adjacent to the anterior flexible lens is then bonded 360° around (at the opening of the capsulorrhexis) by the surgeon to the anterior capsule to secure the lens in place. This approach has numerous drawbacks, a few of which follow. First, several aspects of the procedure are substantially difficult and not within the technical skill level of many eye surgeons. For example, creation of the desired round capsullorrhexis within the stated tolerance required is particularly difficult. Second, the bonding “ledge” may disrupt the optical image produced by the adjacent optic. Third, intraocular bonding requires a high degree of skill, and may fail if the capsullorrhexis is not 360° round. Fourth, the proposed method invites cautionary speculation as to the result should the glue fail to hold the lens in position in entirety or over a sectional region. Fifth, it is well known that after lens implantation surgery the capsular bag, upon healing, shrinks. Such shrinking can distort a lens glued to the bag in a pre-shrunk state, especially since the lens is permanently affixed to a structure which is not yet in equilibrium. Sixth, Thompson fails to provide a teaching as to how or when to release the gel from the expansion channel; i.e., remove the stress from the lens. If the gel is not removed, the lens will not accommodate. If the gel is removed during the procedure, the lens is only in a flattened non-accommodating shape during adhesion to the capsule, but not post-operatively, and it is believed that the lens therefore will fail to interact with the ciliary body as required to provide the desired accommodation as the capsular bag may change shape in the post-operative period. If the gel is otherwise removed thereafter, Thompson ostensibly requires an additional surgical procedure therefor. In view of these problems, it is doubtful that the lens system disclosed by Thompson can be successfully employed.

Thus, the prior art discloses numerous concepts for accommodating intraocular lenses. However, none are capable of providing an accommodating implant which does not, in one way or another, risk damage to the ciliary body or the capsular bag, present technical barriers, or present potential serious consequences upon failure of the device.

### SUMMARY OF THE INVENTION

It is therefore an object of the invention to provide an intraocular lens that functions similarly to the natural crystalline lens.

It is another object of the invention to provide an intraocular lens that changes shape and increases power during accommodation.

It is also an object of the invention to provide an intraocular lens that produces a sufficient increase in focusing power such that it is clinically useful.

It is an additional object of the invention to provide an intraocular lens that permits uncomplicated implantation of the lens in a manner compatible with modern-day cataract surgery techniques.

In accord with these objects, which will be discussed in detail below, an intraocular lens (IOL) system that permits accommodation and a method of implanting such an intraocular lens system are provided. According to one embodiment of the invention, the intraocular lens system includes a flexible optic having a skirt (periphery or haptic), and a restraining element about the skirt and adapted to temporarily maintain the flexible optic in a stressed, non-accommodating configuration during a post-operative period. The retraining element may comprise a dissolvable bioabsorbable material such that the element automatically releases the optic after a post-operative period, or may be released under the control of a eye surgeon, preferably via a non-surgically invasive means such as via a laser or a chemical agent added to the eye.

According to a preferred method of implantation, the ciliary body muscle is pharmacologically induced into a relaxed stated (cycloplegia), a capsulorrhexis is performed on the lens capsule, and the natural lens is removed from the capsule. The prosthetic lens is then placed within the lens capsule. According to a preferred aspect of the invention, the ciliary body is maintained in the relaxed state for the duration of the time required for the capsule to naturally heal and shrink about the lens; i.e., possibly for several weeks. After healing has occurred, the restraining element automatically or under surgeon control releases the optic from the stressed state. The ciliary body and lens may then interact in a manner substantially similar to the physiological interaction between the ciliary body and a healthy natural crystalline lens.

Alternatively, a fully relaxed lens (i.e., without restraining element) can be coupled to a fully stressed and contracted ciliary body.

The intraocular lens system of the invention is compatible with modern cataract surgery techniques and allows for large increases in optical power of the implanted lens. Unlike other proposed accommodating intraocular lens systems, the lens described herein is capable of higher levels of accommodation and better mimics the function of the lens of the human eye. Further, unlike other lens systems previously described, the lens take into account certain reciprocal aspects of the relationship between the natural crystalline lens and the ciliary body. Moreover, the implantation is relatively easy and rapid.

Additional objects and advantages of the invention will become apparent to those skilled in the art upon reference to the detailed description taken in conjunction with the provided figures.

### BRIEF DESCRIPTION OF THE DRAWINGS

FIG. 1 is a diagrammatic view of a cross-section of a normal eye;

FIG. 2 is a graph of the stresses on the ciliary body-crystalline lens system of the eye in a continuum of states between distance vision and full accommodation;

FIG. 3 is a schematic front view of an intraocular lens according to the invention configured into a stressed state with a restraining element;

FIG. 4 is a schematic transverse section view of the intraocular lens of FIG. 3 in a stressed state;

FIG. 5 is a schematic transverse section view of the intraocular lens of FIG. 3 in a non-stressed accommodating state;

FIGS. 6 and 7 are other schematic transverse section views of intraocular lenses according to the invention;

FIG. 8 is a schematic front view of an intraocular lens according to the invention with the restraining element removed, and thus, configured in a non-stressed accommodating state;

FIG. 9 is a transparent front view of an intraocular lens according to the invention shown with a second embodiment of a restraining element;

FIG. 10 is a schematic transverse view of the intraocular lens of FIG. 9;

FIG. 11 is a transparent front view of an intraocular lens according to the invention shown with a third embodiment of a restraining element;

FIG. 12 is a schematic transverse view of the intraocular lens of FIG. 11;

FIG. 13 is a transparent front view of an intraocular lens according to the invention shown with a third embodiment of a restraining element;

FIG. 14 is a schematic transverse view of the intraocular lens of FIG. 13;

FIG. 15 is a schematic front view of an intraocular lens according to the invention having a particular skirt configuration which include haptics and another alternate embodiment restraining element;

FIG. 16 is a schematic front view of another intraocular lens according to the invention having a particular skirt configuration which include haptics and yet another alternate embodiment restraining element;

FIG. 17 is a schematic side view of the intraocular lens of FIG. 16;

FIG. 18 is an intraocular lens according to the invention having a particular skirt configuration which include haptics and yet a further alternate embodiment restraining element;

FIG. 19 is a block diagram of a first embodiment of a method of implanting an intraocular lens according to the invention;

FIG. 20 is a block diagram of a second embodiment of a method of implanting an intraocular lens according to the invention; and

FIG. 21 is a block diagram of a third embodiment of a method of implanting an intraocular lens according to the invention.

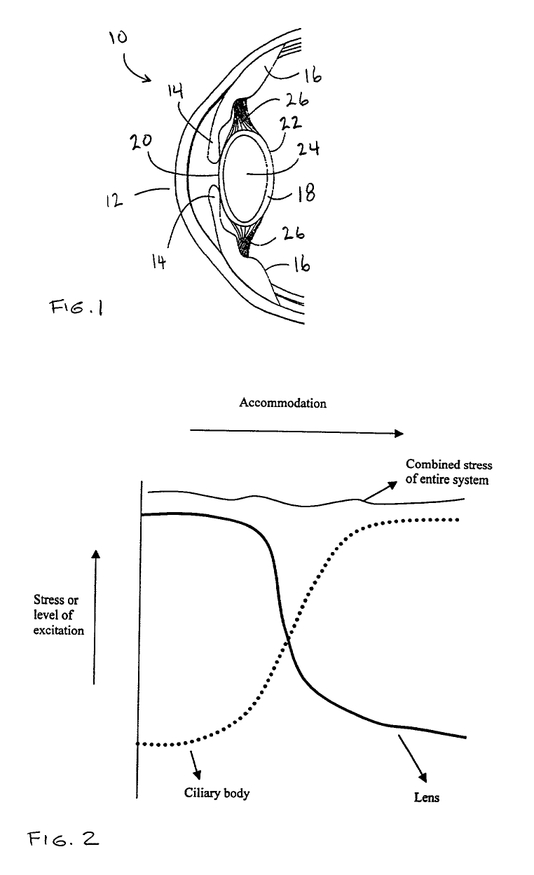

### DETAILED DESCRIPTION OF THE PREFERRED EMBODIMENTS

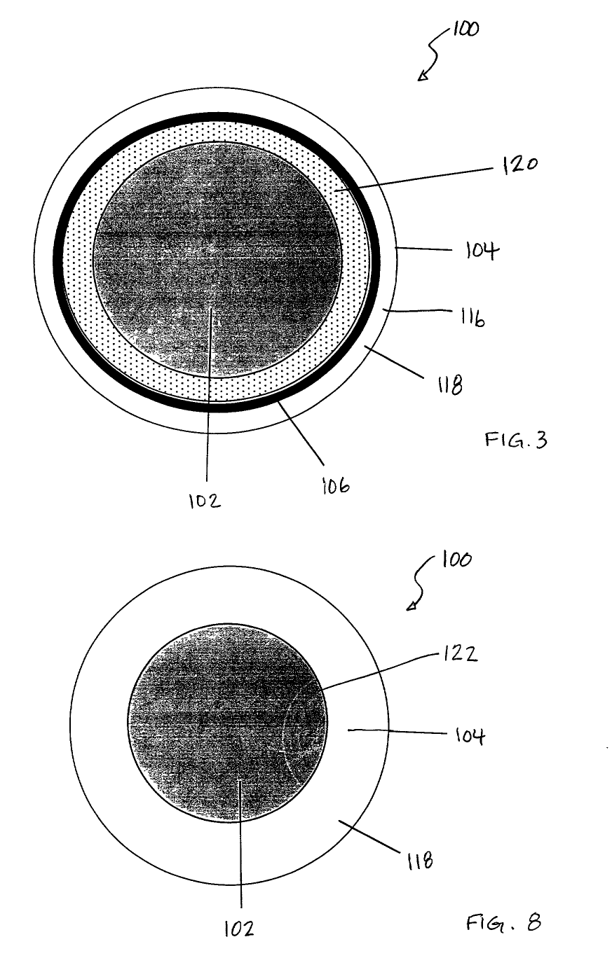

Turning now to FIG. 3, a first preferred embodiment of an intraocular lens 100 according to the invention is shown. The lens includes a pliable optic portion 102 having an elastic memory, and is peripherally surrounded by a skirt portion 104. A restraining element 106 is provided on the skirt portion 104 and operates to hold the skirt portion and optic portion 102 in a stressed (i.e., stretched) configuration. Comparing FIG. 3, showing the optic portion in a stressed configuration, with FIG. 8, showing the optic portion in a non-stressed configuration, it is seen that the optic portion has a smaller diameter in the non-stressed configuration.

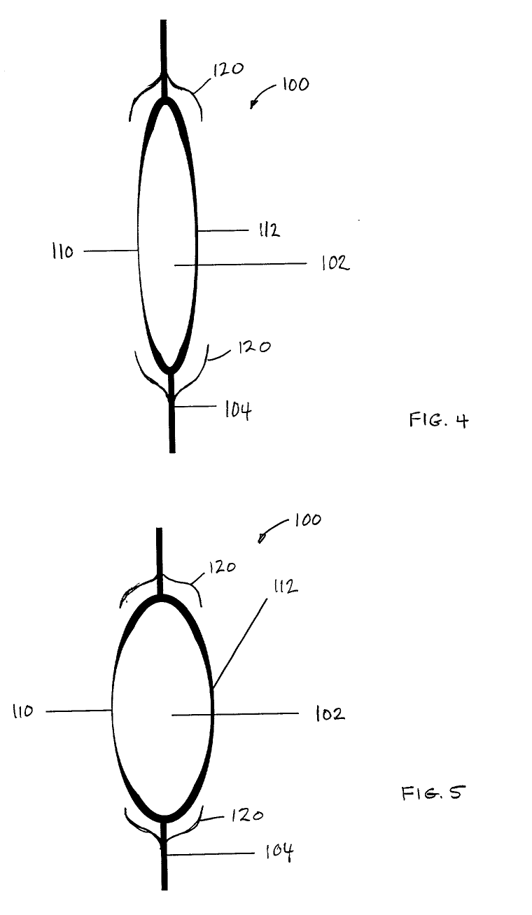

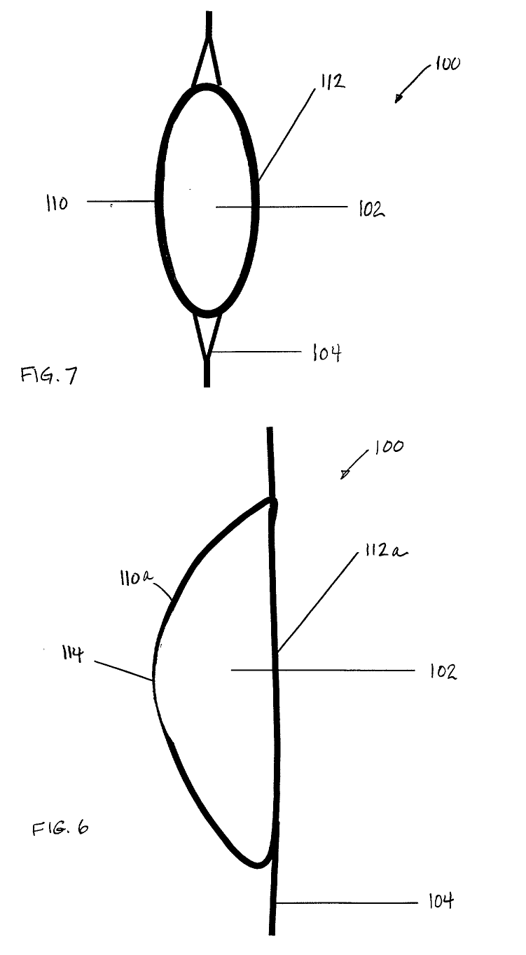

More particularly, the optic portion 102 is typically approximately 5 to 6 mm in diameter and made from a silicone polymer or other suitable flexible polymer. The optic portion defines an anterior surface 110 and a posterior surface 112. The optic portion may have a biconvex shape in which each of the anterior surface 110 and posterior surface 112 have similar rounded shapes. FIG. 4 illustrates such a lens in a stressed non-accommodating configuration, while FIG. 5 illustrates such a lens in the non-stressed accommodating configuration. Alternatively, referring to FIG. 6, the anterior surface 110a may be provided with a substantially greater curvature than the posterior surface 112a. In addition, referring to FIG. 7, the anterior and posterior surfaces 110, 112 of the optic portion can be evenly pliable throughout, or, referring back to FIG. 6, greater flexibility and pliability can be fashioned into the central portion 114 of the anterior 110 surface of the lens to enhance the accommodating effect. This may be done by using materials of differing modulus of elasticity or by altering the thickness of the central portion and/or anterior surface 110 of the optic portion 102.

Referring back to FIG. 3, the skirt portion 104 has substantially less pliability than the optic portion 102. The periphery 116 of the skirt portion 104 is preferably provided with a plurality of circumferentially displaced fenestration holes 118. The fenestration holes 118 operate to promote firm attachment of the capsular bag to the lens skirt 104 during the healing period. That is, during the healing process, the capsular bag shrinks by a substantial amount and portions of the anterior and posterior capsular bag enter into the fenestration holes 118 and join together to lock the lens 100 within the capsule without necessitating any bonding agent, sutures, or the like. Alternatively, the peripheral portion 104 could be fashioned with a textured surface, ridges or any surface modification that promotes strong adhesion of the capsule to the lens skirt 104.

Referring to FIGS. 3 and 4, according to a preferred, though not essential, aspect of the invention, a preferably thin and pliable collar 120 is positioned around the anterior surface of lens near the junction 122 (FIG. 8) of the optic portion 102 and the skirt portion 104 to keep the more central portions of the anterior capsular remnant from adhering to the optic portion. The collar is preferably made from silicone or another smooth polymer.

As discussed above, the skirt portion 104 is maintained in a stressed configuration by the restraining element until the restraining element is removed. According to a preferred embodiment of the restraining element, the restraining element is a band provided on the outside of the skirt portion. The band 106 is preferably comprised of a dissolvable, preferably bioasborbable material that is adapted to preferably naturally dissolve in the fluid of the eye within a predetermined period of time after implantation. Alternatively, the dissolvable material may be selected so that it dissolves only upon the addition of a dissolving-promoting agent into the eye. Preferred dissolvable materials for the restraining band 106 include collagen, natural gut materials, glycan, polyglactin, poliglecaprone, polydioxanone, or other carbohydrate-based or protein-based absorbable material.

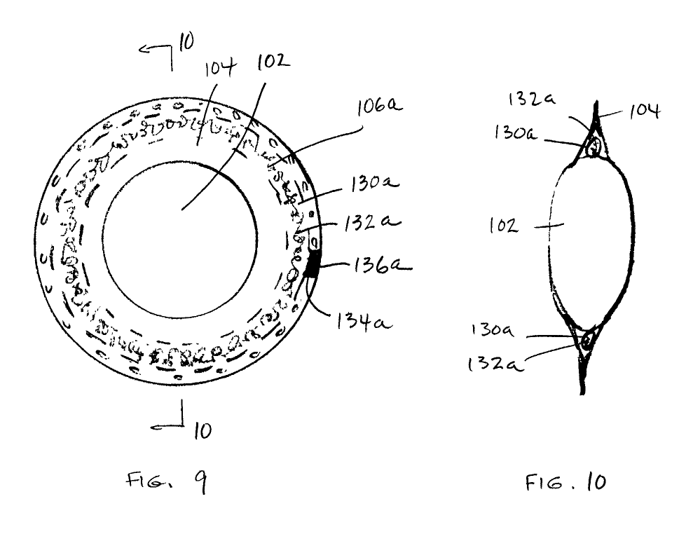

Referring now to FIGS. 9 and 10, according to a second embodiment of the restraining element 106a, the restraining element comprises a circumferential channel 130a in the skirt 104 that is filled with a fluid or gel 132a. Preferably an isotonic solution such as a balanced salt solution is used. Alternatively, other suitable fluids, solution, or gels, including viscoelastics can be used. The channel 130a has an outlet 134a that is blocked by a dissolvable, preferably bioabsorbable seal 136a. The filled channel 130a operates to stress the optic portion 102 into a non-accommodating configuration until the seal 136a is dissolved and the outlet 134a is thereby opened. Then, the material 132a within the channel 130a is forced out of the channel by the natural elasticity of the lens and permits the lens to move in accord with the excitation state of the ciliary body; i.e., between non-accommodative and accommodative states. Alternatively, the seal material 136a may not be naturally dissolvable within the environment of the eye, but rather is dissolvable within the presence of a chemical agent, such as an enzyme, which can be added to the eye. In such case, the eye surgeon can non-surgically control the release of the seal.

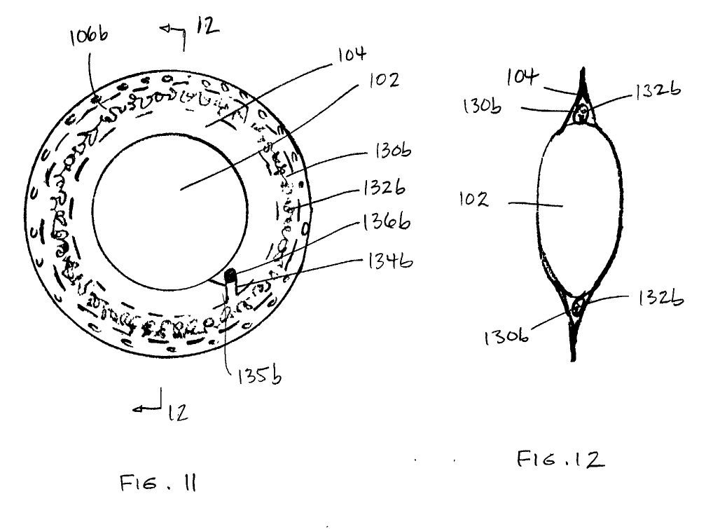

Turning now to FIGS. 11 and 12, according to a third embodiment of the restraining element, the restraining element 106b comprises a circumferential channel 130b in the skirt portion 104 that is filled with a balanced salt solution or other suitable material 132b that maintains the optic portion into a non-accommodating stressed configuration. The channel 130b has an outlet tube 134b that is biased outward from the optic portion 108 but which is preferably anchored with an anchor 135b toward the optic portion 102 but which preferably does not overlie a central area of the optic portion which would interrupt the vision of the patient when the lens is implanted. The outlet tube 134b is provided with a seal 136b made from a material, e.g., hard silicone, polymethylmethacrylate (PMMA) or plastic, that is ablatable or otherwise able to be unsealed by laser light from a YAG laser or other laser suitable for eye surgery. Likewise, the anchor 135b is also made from such a material. When the lens is implanted, as discussed in detail below, the anchor 135b and the outlet tube 134b, by being directed toward the optic portion 102, is visible to the eye surgeon through a dilated iris and is positioned to receive laser light. In this embodiment, the seal 136b can be removed and the outlet tube 134b opened under the full control of the eye surgeon (at his or her discretion upon postoperative evaluation of the lens recipient) by use of a laser to remove the pressure in the channel 130b to equilibrate with the anterior chamber pressure of the eye. Moreover, removal of the anchor 135b enables the outlet tube to move away from the optic portion in accord with its bias and toward the periphery to minimize any potential interference with the patient's vision.

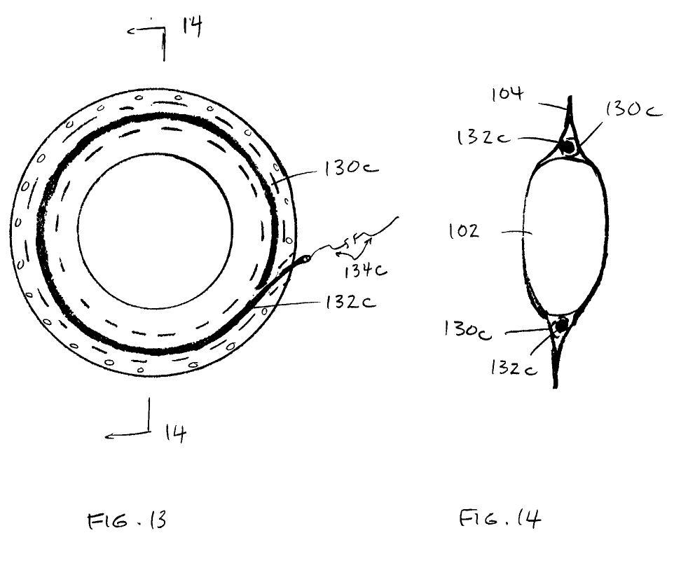

According to a fourth embodiment of the restraining element, any mechanical means for maintaining the lens in a stressed configuration can be used. For example, referring to FIGS. 13 and 14, a relatively stiff restraining element 132c having a circular form can be inserted or otherwise provided within a circumferential channel 130c. The restraining element is made from a material designed to be ablated or broken upon receiving laser energy, e.g., hard silicone, polymethylmethacrylate (PMMA) or plastic. Alternatively, the end of the element 132c can be provided with a length of flexible material 134c, e.g., suture, which can be extended to outside the eye. When it is desired to remove the restraining element, the surgeon grasps the suture with a forceps and pulls the suture. This either removes the restraining element from the lens or breaks the restraining element. In either case, the stress is released from the optic. As yet another less preferred alternative, stiff restraining element is removable or broken only upon an invasive (requiring an incision) surgical procedure.

Other embodiments for the restraining elements and removal thereof are possible. For example, and not by way of limitation, the seal for an inflated channel can be attached to a suture or other length of flexible material which extends outside the eye. The suture can be pulled by the surgeon to remove the seal. In yet another example, shallow shells, adapted to be dissolvable naturally or in conjunction with an additive agent, may be provided to the front and back of the optic portion to force the optic portion to adopt a flatter (i.e., stressed) configuration. By way of another example, dissolvable or laser-removable arced struts may be provided across the lens which force the optic portion into a stressed state.

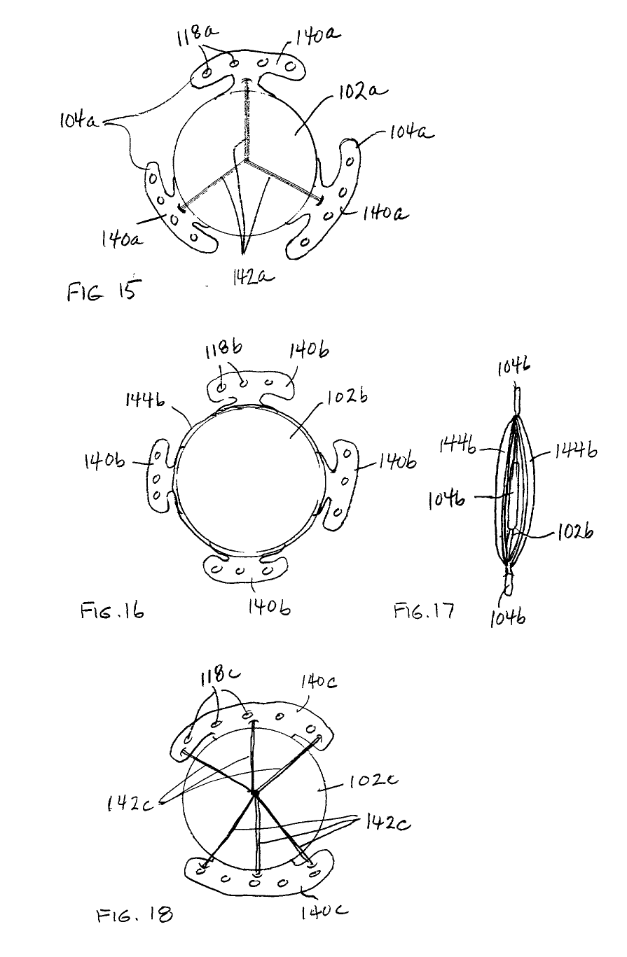

Moreover, embodiments of the restraining element which maintain the stressed state of the optic via external flattening of the optic or by arced struts are suitable for use with a non-circumferential skirt portion; i.e., where the skirt portion is defined by a plurality of haptics extending outward from the optic portion. For example, FIGS. 15-18, illustrate the “skirt portion” defined by a plurality of haptics, rather than a complete ring about the optic. FIG. 15 discloses a skirt portion 104a defined by three haptics 140a, each of which preferably includes fenestration holes 118a. Dissolvable or laser-ablatable arced struts 142a are situated to maintain a radial stress on the optic portion 102a; i.e., the struts 142a function together as a restraining member. FIGS. 16 and 17 discloses a skirt defined by four haptics 140b, each of which preferably includes fenestration holes 118b. Shells 144b are coupled to the haptics anterior and posterior of the optic to flatten the optic. FIG. 18 discloses a skirt defined by two haptics 140c, each of which preferably includes fenestration holes 118c. Multiple struts 142c are coupled to each haptic 140c.

In addition, it is recognized that the optic portion may be provided in an optically transparent bag, and the bag may be pulled or otherwise forced taught to stress the optic. The bag may be pulled taught by using one of the restraining element described above, e.g., retaining rings, channels, shells, or struts, or any other suitable means, provided either directly to the bag or provided to an element coupled about a periphery of the bag.

Moreover, it is recognized that the lens of the invention may comprise two optic elements: one stationary and the other adapted to change shape and thereby alter the optic power of the dual optic system. In such an embodiment, the optic element adapted to change shape would be provided in a stressed-configuration, according to any embodiment described above.

In each embodiment of the restraining element, the restraining element is preferably configured on or in the lens during manufacture, such that the lens is manufactured, shipped, and ready for implant in a fully stressed configuration.

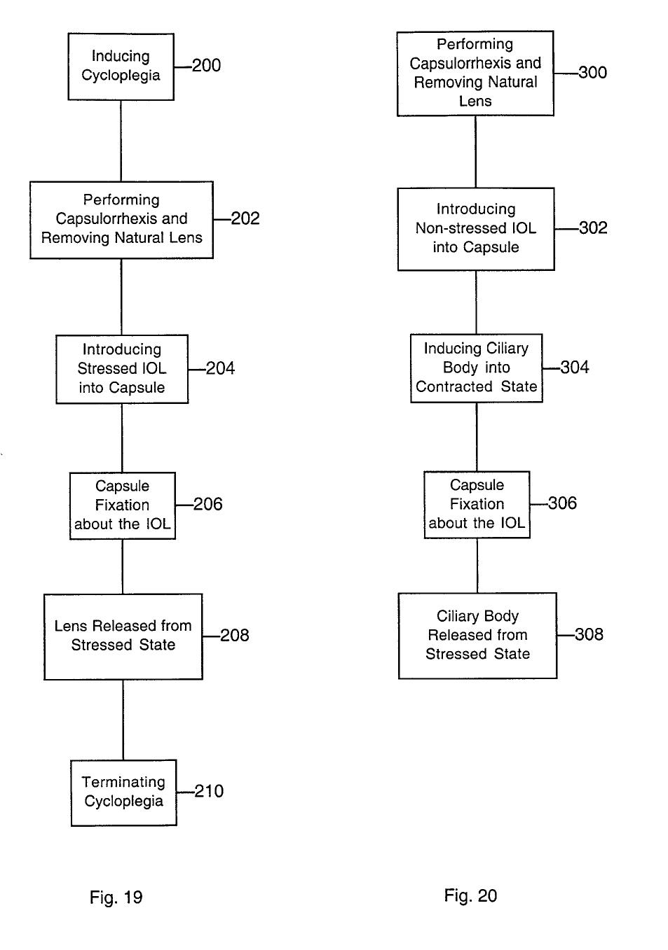

The lens is implanted according to a first method of implantation, as follows. Referring to FIG. 19, the patient is prepared for cataract surgery in the usual way, including full cycloplegia (paralysis of the ciliary body) at 200. Cycloplegia is preferably pharmacologically induced, e.g., through the use of short-acting anticholinergics such as tropicamide or longer-lasting anticholinergics such as atropine.

An anterior capsullorrhexis is then performed at 202 and the lens material removed. A stressed lens according to the invention is selected that preferably has an optic portion that in a stressed-state has a lens power selected to leave the patient approximately emmetropic after surgery. The lens is inserted into the empty capsular bag at 204.

Cycloplegia is maintained for several weeks (preferably two to four weeks) or long enough to allow the capsular bag to heal and “shrink-wrap” around the stressed and elongated lens at 206. This can be accomplished post-operatively through the use of one percent atropine drops twice daily. As the lens shrinks, the anterior and posterior capsular bag walls enter into the fenestration holes and join together to lock the lens in position.

If the lens includes a restraining element having a dissolvable component, eventually the dissolvable material is lost from the lens, and the lens is unrestrained. If the lens includes a restraining element having a laser-removable component, a surgeon may at a desired time remove the component to place the lens in a unrestrained configuration. If the lens includes a retraining element which must be surgical removed or altered, the surgeon may at a desired time perform a second eye procedure to remove the component and place the lens in an unrestrained configuration.

Regardless of the method used, when the lens is unrestrained (i.e., released from the stressed state) at 208 and the post-operative cycloplegia medicines are stopped at 210 the lens is initially still maintained in a stressed state (FIG. 4) due to the inherent zonular stress of the non-accommodating eye. When the patient begins accommodating, the zonular stress is reduced and the implanted lens is permitted to reach a more relaxed globular conformation, as shown in FIGS. 5 and 8. This change in shape provides the optic with more focusing power and thus accommodation for the patient is enabled. As with the natural crystalline lens, the relaxation of the implanted lens to a more globular shape is coupled with a development of strain or stress in the ciliary body during accommodation. Further, when the patient relaxes accommodation, the stress in the ciliary body is reduced, and there is a compensatory gain in stress as the lens is stretched into its non-accommodative shape (See again FIG. 2).

Referring to FIG. 20, according to another embodiment of the method of the invention, a lens of similar design as described above is used, except that there is no restraining element on the lens. Temporary cycloplegia is induced, and a capsulorrhexis is performed 300. The lens is implanted while the ciliary body is in a fully relaxed state at 302. The patient is then fully accommodated (i.e., the ciliary body is placed in a contracted state) at 304, preferably through pharmacological agents such as pilocarpine.

Once the capsular bag is fully annealed (affixed) to the lens periphery at 306, the pharmacological agent promoting accommodation is stopped at 308. Then, as the ciliary body relaxes, the lens is stretched into an elongated shape having less focusing power. Conversely, as accommodation recurs, the lens returns to it resting shape having greater focusing power.

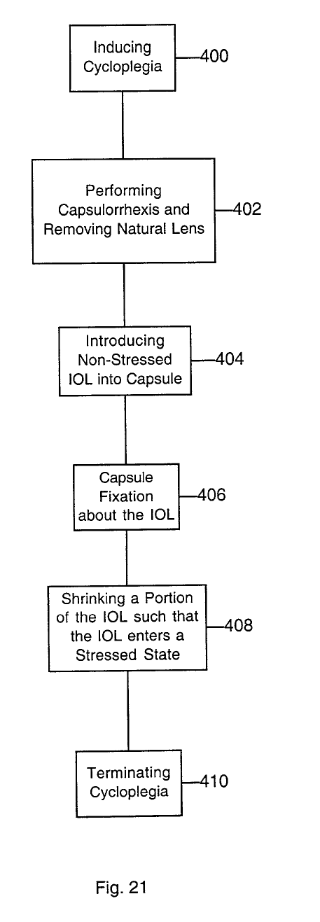

Referring to FIG. 21, in yet another embodiment of the method of the invention, the patient is cyclopleged during cataract surgery at 400, a capsulorrhexis is performed at 402, and a flexible lens in an unstressed state is implanted in the capsular bag at 404. After a few weeks of complete cycloplegia and during which capsular fixation of the lens periphery is accomplished at 406, light (e.g., ultraviolet or infrared), a chemical agent, or another suitable means is used to shrink the optic or the adjacent skirt of the lens while the patient is still fully cyclopleged at 408. In this manner, the optic is again placed into a stressed configuration while the ciliary body is fully relaxed. As with previous embodiments, when cycloplegia is stopped and accommodation occurs at 410, the lens is able to return to a more relaxed globular configuration.

There have been described and illustrated herein several embodiments of an intraocular lens and methods of implanting the same into an eye. While particular embodiments of the invention have been described, it is not intended that the invention be limited thereto, as it is intended that the invention be as broad in scope as the art will allow and that the specification be read likewise. Thus, while two particular states of intraocular lenses (fully stressed and fully accommodating) have been disclosed, it will be appreciated that there is a continuum of states of stress that can be fashioned in the inserted lens that would be appropriate for any given state of the ciliary body. In addition, while particular types of materials have been disclosed for the lens, the dissolving material, and a viscoelastic material (where used), it will be understood that other suitable materials can be used. Also, while exemplar pharmacological agents are disclosed for maintaining a state of the ciliary body, it is understood that other agents can be used. Furthermore, while the skirt has been shown comprised of a two to four haptics, it is recognized that a single haptic or five or more haptics may be utilized. Moreover, while the restraining struts and shells have been described with respect to skirts comprising haptics, it will be appreciated that the restraining struts and shells can be used with a circular skirt, as described with respect to the preferred embodiments. It will therefore be appreciated by those skilled in the art that yet other modifications could be made to the provided invention without deviating from its spirit and scope as claimed.

What is claimed is:

1. An intraocular lens for replacement of a natural crystalline lens within a capsular bag during eye surgery, said lens comprising:
a) an optic portion adapted to focus light, said optic portion having a first flexibility;
b) a peripheral portion about said optic portion, said peripheral portion having a second flexibility less than said first flexibility; and
c) a restraining element provided to said peripheral portion and adapted to maintain said optic portion in a stressed state,
said restraining element adapted to be removed after completion of an eye surgery without an invasive surgical procedure,
wherein upon removal of said restraining element, said optic portion is biased toward an unstressed state.

2. An intraocular lens according to claim 1, wherein:
said restraining element comprises a ring of material on said peripheral portion.

3. An intraocular lens according to claim 2, wherein:
said ring of material is dissolvable.

4. An intraocular lens according to claim 2, wherein:
said material is not naturally dissolvable in the environment of the eye, but is dissolvable in the presence of a dissolving agent.

5. An intraocular lens according to claim 2, wherein:
said ring of material is a carbohydrate-based or protein-based material.

6. An intraocular lens according to claim 2, wherein:
said ring of material is adapted to be broken or removed by laser light to release the optic into said unstressed state.

7. An intraocular lens according to claim 1, wherein:
said peripheral portion includes a channel, and said restraining element includes a fluid placed within said channel,
said channel including an outlet having a seal that prevents said fluid from escaping said channel,
said seal adapted to be opened in a non-surgically invasive manner.

8. An intraocular lens a according to claim 7, wherein:
said seal comprises a dissolvable material.

9. An intraocular lens according to claim 8, wherein:
said seal comprises a material removable upon the application of laser light.

10. An intraocular lens according to claim 9, wherein:
said outlet includes a tubular element that extends toward said optic portion.

11. An intraocular lens according to claim 7, further comprising:
a length of flexible material substantially outside peripheral portion and having two ends, one of said ends being coupled to said seal,
wherein upon pulling said length of material with sufficient force relative to said outlet, said seal is removed from said outlet.

12. An intraocular lens according to claim 7, wherein:
said fluid is a balanced salt solution.

13. An intraocular lens according to claim 1, wherein:
said peripheral portion includes a channel having an outlet,
said restraining element includes a relatively stiff circular element within said channel.

14. An intraocular lens according to claim 13, further comprising:
a flexible length of material substantially outside peripheral portion,
said stiff circular element having two ends, one of which is coupled to said length of material,
wherein upon pulling said length of material with sufficient force relative to said peripheral portion, said stiff circular element is adapted to be broken or withdrawn from said channel.

15. An intraocular lens according to claim 1, wherein:
said restraining element comprises dissolvable or laser-removable shell elements provided external said optic portion at anterior and posterior sides thereof.

16. An intraocular lens according to claim 1, wherein:
said restraining element comprises a plurality of dissolvable or laser-removable struts extending from said peripheral portion and across said optic portion.

17. An intraocular lens according to claim 1, further comprising:
a bag provided about said optic portion,
wherein said peripheral portion is defined by one of said bag and an element provided about said bag.

18. An intraocular lens according to claim 1, wherein:
said peripheral portion includes structure adapted to promote tissue attachment thereto.

19. An intraocular lens according to claim 1, wherein:
said peripheral portion comprises a plurality of haptics.

20. An intraocular lens according to claim 1, further comprising:
d) a collar provided on said peripheral portion and adapted to prevent an anterior portion of the capsular bag from adhering to said optic portion.

21. An intraocular lens for replacement of a natural crystalline lens within a capsular bag during eye surgery, said lens comprising:
a) an optic portion adapted to focus light, said optic portion having a first flexibility;
b) a peripheral portion about said optic portion, said peripheral portion having a second flexibility less than said first flexibility; and
c) a dissolvable restraining element provided to said peripheral portion and adapted to maintain said optic portion in a stressed state until dissolution in an environment of the eye wherein said optic portion is then biased toward an unstressed state.

22. An intraocular lens for replacement of a natural crystalline lens within a capsular bag during eye surgery, said lens comprising:
a) an optic portion adapted to focus light, said optic portion having a first flexibility;
b) a peripheral portion about said optic portion, said peripheral portion having a second flexibility less than said first flexibility;
c) a restraining element provided to said peripheral portion in a restraining configuration such that said restraining element is adapted to maintain said optic portion in a stressed state; and
d) a dissolvable or laser-removable element which upon dissolution or removal releases said restraining element from said restraining configuration and permits said optic portion to enter an unstressed state.

23. A method of implanting an intraocular lens into an eye, comprising:
a) inducing cycloplegia;
b) providing an intraocular lens in a stressed state;
c) inserting the intraocular lens into a capsular bag of the eye;
d) maintaining cycloplegia until the capsular bag physiologically affixes to the intraocular lens; and
e) releasing the intraocular lens from the stressed state.

24. A method according to claim 23, wherein
said releasing is non-surgically invasive.

25. A method according to claim 24, wherein:
said intraocular lens includes a restraining means for restraining said lens in the stressed state, said releasing including providing an agent to the eye which operates to dissolve said restraining means.

26. A method of implanting an intraocular lens into an eye, comprising:
a) providing an intraocular lens in a relaxed non-stressed state;
b) inserting the intraocular lens into a capsular bag of the eye;
c) inducing a ciliary body of the eye into a contracted state; and
d) maintaining the ciliary body of the eye in the contracted state until the capsular bag physiologically affixes to the intraocular lens.

27. A method according to claim 26, wherein:
said ciliary body is pharmacologically induced into the contracted state.

28. A method of implanting an intraocular lens into an eye, comprising:
a) inducing cycloplegia;
b) providing an intraocular lens in an unstressed state;
c) inserting the intraocular lens into a capsular bag of the eye;
d) maintaining cycloplegia, during which the capsular bag physiologically affixes to the intraocular lens; and
e) after the capsular bag physiologically affixes and during cycloplegia, changing a size of said lens such that the lens enters into a stressed state.

29. A method according to claim 28, wherein:
said lens is changed in size by shrinking a portion of the lens.

30. A method according to claim 29, wherein:
light is used to shrink the portion of the lens.

31. A method according to claim 29, wherein:
a chemical agent is used to shrink the portion of the lens.

32. A method according to claim 28, further comprising:
f) terminating cycloplegia.
